(19) United States
(12) Patent Application Publication
(10) Pub. No.: US 2001/0051143 A1
(43) Pub. Date: Dec. 13, 2001

(54) KERATIN TREATING COSMETIC COMPOSITIONS CONTAINING HIGH DS CATIONIC GUAR GUM DERIVATIVES

(76) Inventors: IAN W. COTTRELL, PRINCETON, NJ (US); GARY T. MARTINO, JAMESBURG, NJ (US); KIMBERLY A. FEWKES, SKILLMAN, NJ (US)

Correspondence Address: EUGENE ZAGARELLA JR, NATIONAL STARCH AND CHEMICAL COMPANY, BOX 6500, BRIDGEWATER, NJ, 088070500

(21) Appl. No.: 09/299,168

(22) Filed: Apr. 26, 1999

(51) Int. Cl. A61K 7/42; A61K 7/06; A61K 7/075; A61K 7/08; C07H 1/00; C13K 1/00; A61K 7/00; C08B 37/00; C07G 17/00; C07H 3/00; A61K 7/11; C07H 13/02; C07H 15/04; C07H 1/06; C07H 1/08; A61K 6/00

(52) U.S. Cl. 424/70.28; 424/70.1; 424/70.11; 424/70.13; 424/59; 424/401; 536/124; 536/120; 536/127; 536/119; 536/123.1

(57) ABSTRACT

A compatible cosmetic composition for treating keratin substances comprising a cationic guar gum which contains a cationic group selected from the group consisting of amino, ammonium imino, sulfonium or phosphonium groups and wherein the cationic content is represented by a DS of from about 0.25 to 1.0 and an effective surfactant amount of at least one non-ionic or anionic surfactant.

### BACKGROUND OF THE INVENTION

This invention relates to compatible cosmetic compositions used for treating keratin substances and which contain selected high DS (degree of substitution) cationic guar gum derivatives. The resulting compositions have good compatibility and provide excellent performance including clarity, substantivity to hair and skin and wet comb performance on hair.

Various disclosures of cationic and anionic derivatives of polymers, polysaccharides and polygalactomannans have been made for a number of applications and uses. Cosmetic compositions containing polymer, polysaccharide and polygalactomannan derivatives for use in treating keratin substances such as hair, skin and nails are also known.

Shampoo compositions containing cationic derivatives of galactomannan gum along with an anionic surfactant and a water-insoluble sunscreen are disclosed in U.S. Pat. No. 5,186,928 issued on Feb. 16, 1993 to D. Birtwistle. U.S. Pat. No. 5,387,675 issued Feb. 7, 1995 to M. Yeh discloses cationic quaternary ammonium ethers of polysaccharides which contain a hydrophobic group and are useful as thickening agents in different applications. Cationic organic polymers are disclosed as conditioning agents in hair shampoos in U.S. Pat. No. 5,573,009 issued Nov. 12, 1996 to R. Wells. Cationic starches of the quaternary ammonium type are disclosed as demulsifiers in U.S. Pat. No. 4,088,600 issued May 9, 1978 to T. Tutein et al. and Japanese Patent Disclosure 55-45602, published Mar. 31, 1980 to T. Yanagikawa et al. shows cationized hydroxyalkyl starches used in cosmetic compositions.

Despite the various disclosures of cationic and other derivative for use in cosmetic composition, problems of compatibility and other performance charactertistics often arise in certain compositions, particularly those containing non-ionic and amphoteric surfactants. Accordingly, the need exists for cosmetic keratin treating compositions which are compatible while providing desirable performance attributes such as clarity and substantivity.

### SUMMARY OF THE INVENTION

This invention is directed to compatible cosmetic compositions for treating keratin substances and which contain selected high DS (degree of substitution) cationic guar gum derivatives and one or more non-ionic and/or amphoteric surfactants. These selected cationic guar gum derivatives are prepared using guar gum powder in an alcohol solvent process followed by purification with base.

More particularly, this invention involves a compatible composition for treating keratin substances comprising a cationic guar gum derivative which contains a cationic group selected from the group consisting of amino, ammonium, imino, sulfonium or phosphonium groups and wherein the cationic content is represented by a DS of from about 0.25 to 1.0, and an effective surfactant amount of at least one non-ionic and/or amphoteric surfactant. The cationic guar gum derivative as used in the composition of this invention is prepared by reacting guar gum with the cationic reagent in an alcohol solvent medium followed by purification of the reaction product with base.

### DETAILED DESCRIPTION OF THE INVENTION

The composition of this invention comprises a selected high DS cationic polygalactomannan derivative. The polygalactomannan base material is composed principally of galactose and mannose units and are usually found in the endosperm of leguminous seeds. Guar, tara and locust bean are illustrative polygalactomannans with guar gum being the preferred material for use in this invention.

The polygalactomannan or guar gum material is derivatized or modified to contain a cationic group. The cationic group may be an amino, ammonium, imino, sulfonium or phosphonium group. Such cationic derivatives include those containing nitrogen containing groups comprising primary, secondary, tertiary and quaternary amines and sulfonium and phosphonium groups attached through either ether or ester linkages. Particularly useful cationic derivatives are those containing amino or nitrogen groups having alkyl, aryl, alkaryl or cyclic substituents of 1 to 8 carbon atoms and especially alkyl of 1 to 6 carbon atoms. The preferred cationic reagents are those containing the tertiary amino and quaternary ammonium ether groups.

The amount of cationic substituent on the high DS cationic polygalactomannan will vary between a degree of substitution (DS) of from about 0.25 to 1.0 and preferably from about 0.25 to 0.6. The term “degree of substitution” (DS) as used herein indicates the average number of sites per sugar unit (galactose or mannose) on which there are substituent units.

While there have been various disclosures of cationic polysaccharides, the ability to provide high DS cationic materials which are compatible and provide suitable performance properties in cosmetic systems has been lacking. Now, it has been found that using the alcohol powder process as described herein, high DS cationic polygalactomannans can be prepared which are compatible and especially useful in cosmetic compositions such as shampoos.

The cationic polygalactomman derivatives and particularly guar gum derivatives are prepared by reacting guar gum in dry or powder form with the cationic reagent in an alcohol medium followed by treatment or purification with alkaline base. The alcohol medium is an aqueous alcohol slurry which provides sufficient water to carry out the reaction and provide at least slight swelling of the polygalactomannan or guar gum while at the same time maintain the integrity of the guar particles. An amount of water of up to 30% by weight based on the total weight of the aqueous solvent system may be used in carrying out this process.

The alcohol medium or solvents that are used are monohydric alcohols of 2 to 4 carbon atoms such as ethanol, isopropyl alcohol, n-propanol and tertiary butanol with isopropyl alcohol being preferred. The alkaline base that is used in this process is alkali metal hydroxide or ammonium hydroxide preferably sodium hydroxide. The amount of alkaline base used can vary from about 10 to 100% and preferably from about 20 to 50% by weight, based on the weight of polygalactomannan or guar gum utilized.

The amount of cationic reagent used in the process of this invention will be a sufficient amount to provide the amount or degree of substitution (DS) desired. More particularly, from about 5 to 400% by weight of the cationic reagent and preferably from about 10 to 300% based on the weight of polygalactomannan or guar gum will be used in this process.

It is noted that the cationic guar gum derivatives prepared by this method are capable of providing relatively clear solutions when added to water. They provide light transmission of greater than 90% at 500 to 600 nanometers when added to water in a 1.0% aqueous solution. This ability to provide clear solutions with these high DS guar derivatives is particularly advantageous in different applications such as cosmetics.

The cationic high DS polygalactomannan derivatives as defined herein are especially useful in cosmetic compositions for the treatment of hair, skin and nails where they provide good compatibility and substantivity as well as other desirable performance characteristics. The use of the selected cationic guar gum derivatives has been found especially suitable in personal cleansing compositions such as shampoo formulations and liquid body washes where they provide compatible systems when using a variety of additives including non-ionic and amphoteric surfactants. These cosmetic compositions have been found to provide excellent performance characteristics including good substantivity without excessive build up, good clarity and good wet comb performance on hair. The keratin treating cosmetic composition of this invention may involve different media or systems and will comprise a suitable cosmetic vehicle or base for the composition. This vehicle may be an aqueous system, a solvent system, a combination of aqueous and solvent systems or an emulsion.

The use of an aqueous system in the cosmetic compositions containing the selected cationic polygalactomannans in accordance with this invention are found in shampoos, topical sprays, hair sprays, gels and mousses, dental care products and products containing fragrances and/or antimicrobial agents. The aqueous system will comprise the selected cationic derivative, surfactants, active additives and functional ingredients, optionally a propellant and the balance water.

Generally an aqueous system will comprise from about 10 to 99.8% by weight water, from about 0.1 to 20% by weight of the cationic derivative, from about 0.1 to 40% by weight of surfactant, from about 0.1 to 30% by weight of active additives and ingredients and from about 0 to 50% by weight of propellant. Preferably the composition will comprise about 50 to 80% by weight water, about 0.2 to 6% by weight of the cationic derivative, about 2 to 30% by weight of surfactant, about 0.1 to 20% by weight of active additives and ingredients and about 0 to 40% by weight of propellant. For dental care products, the aqueous system will comprise from about 10 to 50% by weight water, from about 0.1 to 10% by weight of cationic derivative and from about 40 to 90% by weight of active additives and ingredients.

Various other additives and active and functional ingredients may be included in the cosmetic composition as defined herein. This includes but is not limited to emollients, humectants, thickening agents, surfactants, UV light inhibitors, fixative polymers, preservatives, pigments, dyes, colorants, alpha hydroxy acids, aesthetic enhancers such as starch, perfumes and fragrances, film formers (water proofing agents), antiseptics, antifungal, antimicrobial and other medicaments and solvents. Additionally, the cationic polygalactomannan or guar gum as found in this invention may be used in blends with other conditioning polymers and conditioning agents such as cationic hydroxyethyl cellulose, cationic synthetic polymers and cationic fatty acid derivatives. These blended materials help to provide more substantivity and effective conditioning properties in hair.

Surfactants which are useful in this invention include non-ionic and amphoteric surfactants. Non-ionic surfactants which may be used include polyoxyethyleneated, polyoxypropyleneated or polyglycerolated alcohols, alkylphenols and fatty acids with a linear fatty chain containing 8 to 18 carbon atoms and usually 2 to 30 mols of ethylene oxide, fatty acid amides, alkoxylated fatty alcohol alcohol amines, fatty acid esters, glycerol esters, alkoxylated fatty acid esters, sorbitan esters, alkoxylated sorbitan esters, alkylphenol alkoxylates, aromatic alkoxylates and alcohol alkoxylates. Also useful are copolymers of ethylene oxide and propylene oxide, condensates of ethylene oxide and propylene oxide with fatty alcohols, polyoxyethyleneated fatty amides or amines, ethanolamides, fatty acid esters of glycol, oxyethyleneated or non-oxyethyleneated fatty acid esters of sorbitan, fatty acid esters of sucrose, fatty acid esters of polyethylene glycols, phosphoric acid triesters and fatty acid esters of glucose derivatives.

Among the amphoteric surfactants which can be used are alkylamino- monopropionates, alkylaminodipropionates, betaines such as N-alkylbetaines, N-alkylsulfobetaines, n-alkylamidobetaines, sultaines, glycinates, cycloimidium compounds such as alkylimidazolines, asparagine derivatives and aminopropionates.

Surfactants of the above type are further described and illustrated in U.S. Pat. No. 5,089,252 issued Feb. 18, 1992 to J. Grollier et al.; U.S. Pat. No. 5,186,928 issued Feb. 16, 1993 to D. Birtwistle and U.S. Pat. No. 5,573,709 issued Nov. 12, 1996 to R. Wells. The above noted patents, i.e., '252, '928, and '709 are hereby incorporated by reference.

The topical sprays include the non-aerosol sprays and the aerosol sprays or products containing a propellant. The non-aerosol spray compositions contain no propellant and make use of a mechanical activation device to atomize the composition into a spray. While any of the known propellants may be used in the aerosol compositions of this invention, preferred propellants include the non-halogenated hydrocarbons such as C₃-C₆ straight and branched chain hydrocarbons, i.e., propane, butane, isobutane and mixtures thereof. Other preferred propellants include the ethers, such as dimethyl ether, hydrofluorocarbon and the compressed gases such as air, N₂ and CO₂.

The use of a solvent system as the vehicle or base involves other cosmetic compositions containing the selected cationic polygalactomannan derivative. The solvent system will comprise the selected cationic derivative, active additives and functional ingredients, optionally a propellant and the balance solvents. The solvent may be any of the known organic solvents which may solubilize or disperse components of the composition and more particularly aliphatic alcohols, esters, ethers, ketones, amines and hydrocarbons including the aromatic, nitrated and chlorinated hydrocarbons. Particularly preferred organic solvents are the lower aliphatic alcohols such as the C₁₋₃ alcohols and especially ethanol. Generally the solvent system will comprise from about 5 to 55% by weight of solvent, from about 0.1 to 20% by weight of the cationic derivative, from about 0.1 to 40% by weight of surfactant, from about 0.1 to 25% by weight of additives and ingredients, from about 0 to 75% by weight of propellant and from about 0 to 40% by weight of water. Preferably the composition will comprise about 10 to 30% by weight of solvent, about 0.2 to 6% by weight of cationic derivative, about 2 to 30% by weight of surfactant, about 0.1 to 15% by weight of additives and ingredients, about 0 to 40% by weight of propellant and about 0 to 40% by weight of water. The surfactants, additives and other ingredients and propellants used in the solvent systems are the same as described above for the aqueous systems.

Emulsions may also be used as the vehicle or base for the cosmetic compositions of this invention and products of this type include creams and lotions. These emulsions which comprise water-based and oil-based phases, may be oil-in-water emulsions having oil as the dispersed phase and water as the continuous phase or they may be water-in-oil emulsions with water dispersed in oil, which is the continuous phase. The oil phase, which may comprise from about 10 to 90% by weight of the composition, is typically made up of cosmetically acceptable or conventional oily substances that are soluble in this phase, such as oils, waxes and emulsifiers. Compounds which can be included in the oil phase are typically mineral, animal and vegetable oils and fats, synthetic esters, fatty acids and esters, aliphatic alcohols, higher fatty alcohols, alkyl amines, waxes, so called mineral fats and oils such as paraffin oil, petrolatum, ceresin, silicone oils, silicone fats and fragrances. The water phase may comprise from about 10 to 90% by weight of the composition and this will include water and water soluble components such as alkalis, alkanolamines, polyhydric alcohols and preservatives. These emulsions include one or more emulsifiers which usually are contained in the oil phase but in some instances, depending on the type, may be in the water phase. Emulsifiers which can be used may be ionic or nonionic, are well known and constitute a large group of conventional and commercially available products. They are often characterized by their hydrophilic-lipophilic balance (HLB). Oil-in-water (O/W) emulsifying agents typically have an HLB of more than 6.0 and produce emulsions in which the continuous phase is hydrophilic and such emulsions are generally dispersible in water. Emulsifiers of this type include PEG 300 distearate, sorbitan monolaurate and triethanolamine stearate. Water-in-oil (W/O) emulsifiers usually have an HLB of less than 6.0, preferably below 5, and produce emulsions in which the continuous phase is lipophilic. Such emulsifiers include lanolin, alcohols, ethylene glycol monostearate, sorbitan mono-oleate and PEG 200 dilaurate. Emulsifiers with HLB's of between 5 and 7 may function as either W/O or O/W emulsifiers depending on how they are used.

The amount of emulsifiers used in the emulsion compositions of this invention can vary depending on the system and typically will be an effective emulsifying amount. More particularly, the amount of emulsifier can vary from about 0.1 to 25% by weight of the composition and preferably from about 0.2 to 10%. The emulsion compositions will also contain from about 0.1 to 20% by weight of the cationic derivative, preferably from about 0.2 to 6%; from about 0.1 to 40% by weight of surfactant, preferably from about 2 to 30%; and from about 0.1 to 25% by weight of additives and ingredients, preferably 0.1 to 15%. The surfactants, additives and other ingredients which may be included in the emulsion compositions are the same as those described above for the aqueous systems.

The following examples will further illustrate the embodiments of this invention. In these examples all parts are given by weight and all temperatures in degrees Celsius unless otherwise noted.

### EXAMPLE I

A derivatized cationic guar gum was prepared in the following manner. A 100 g sample of guar gum in powdered form was added to an aqueous mixture of 340 g of isopropyl alcohol and 71.8 g of deionized water in a 3-L glass resin kettle equipped with SS stirrer, condenser, addition funnel and nitrogen inlet. Nitrogen is purged through the system and the contents are mixed for 10 minutes at 200 to 250 rpm. One hundred (100) g of 1,2-epoxypropyl N,N,N-trimethyl ammonium chloride (72.3% aqueous solution) was added via an addition funnel over 15 minutes and stirred for an additional 15 minutes. Sodium hydroxide (18.8 g) (25% aqueous solution) was added over 5 minutes and the reaction mixture stirred for an additional 10 minutes. The reaction temperature was raised to 60° C. (over about 30 minutes) and stirred at this temperature for 3 hours. About 46.9 g of sodium hydroxide (50% aqueous solution) was then added over 30 minutes and the temperature was increased to 70° C. over 30 minutes and held at this temperature with stirring for 1.5 hours. The reaction mixture was cooled to 20 to 25° C. followed by the addition of 200 g of isopropanol and then neutralized with 46.5 g of glacial acetic acid and stirred for 5 to 10 minutes. The slurry was filtered and washed twice with 415 g of aqueous isopropyl alcohol (85%) followed by 400 g of pure isopropyl alcohol. The wet cake was filtered and spread onto an aluminum tray and air dried for 15 hours followed by drying in an oven at 45° C. for 4 hours.

The resulting dry mass was ground and sieved to recover a fine powder of derivatized guar. The product analyzed for a DS of 0.32 on the guar.

A similar procedure was used to prepare other guar derivatives as shown in Example III having different quaternary ammonium substitution levels by varying the amount of cationic reagent.

### EXAMPLE II

A cosmetic shampoo composition was formulated containing a cationic guar gum derivative with a hydroxypropyl trimethyl ammonium cationic group (DS of 0.32) prepared as in Example I. The shampoo composition had the following ingredients:

| Ingredients | Use Levels (% active) |
|---|---|
| PEG Sorbitan Laurate | 10.25 |
| Sodium Trideceth Sulfate | 2.25 |
| Cocamidopropyl Betaine | 4.00 |
| Glycerin | 2.50 |
| Lauroamphodiacetate | 1.12 |
| PEG 150 Distearate | 2.00 |
| Sodium Laureth-13 Carboxylate | 0.50 |
| Dowicil (quaternium-15) | 0.10 |
| EDTA | 0.10 |
| Cationic guar derivative | 0.50 |
| Deionized water | qs |
| | 100.0 |

This shampoo formulation was evaluated for clarity using a Hach Turbidity Meter and had a turbidity of 6.89 Ntu (lower Ntu value represent better clarity with a value of 12 Ntu or lower being visibly clear to the naked eye).

A lumicrease wool dye test was used to determine adsorption and retention of the cationic guar derivative contained in the shampoo composition described above. The method consists of applying the sample formulation to a wool swatch one time (1×) and ten times (10×) and then applying an anionic dye that is attracted to the positively charged polymer. Through color intensity differences, one can determine the relative amount of cationic polymer after washing. Color intensity was determined with a Technidyne Inc. Brightmeter with lower values representing less adsorption to the substrate. The higher the a-value, the more red color and polymer present. Build-up is characterized after 10 washes by comparing initial and final a-values. The shampoo formulation described above had an a-value of 15.69 after 1 wash and 16.77 after 10 washes.

The sample shampoo formulation was evaluated for wet comb performance. The wet comb performance was determined objectively by applying the test shampoo to a damaged blonde hair tress and measuring the resistance in force to pull the comb through the hair. The measurement was made using a MTS Systems Inc. Synergie 200 tensile tester. The percent (%) improvement value indicates the difference between untreated wet hair and hair treated with cationic guar derivative after being rinsed off with water. The value for above described shampoo formulation was 73.5% improvement. This result is shown in Table 1.

### EXAMPLE III

The shampoo composition described in Example II was formulated with different cationic guar derivatives and evaluated for clarity (turbidity), substantivity (adsorption and retention) and build-up, and wet comb performance as described in Example II. The results are given below in Table 1.

TABLE 1

| | | | | Color | | | |
|---|---|---|---|---|---|---|---|
| | | | | Intensity | | | |
| | | | Turb- | (a-value) | | | Wet Comb |
| | Add- | | idity | 1× | 10× | Build- | % Im- |
| Sample | itive | DS | (Ntu) | Wash | Wash | up | provement |
| A | Cationic | 0.32 | 6.9 | 15.69 | 16.77 | 1.08 | 73.5 |
| | Guar | | | | | | |
| B | Cationic | 0.46 | 6.5 | 15.84 | 20.27 | 4.43 | 55.27 |
| | Guar | | | | | | |
| C | Cationic | 0.17 | >100 | 16.45 | 16.7 | 0.25 | 67.9 |
| (Compa- | Guar¹ | | | | | | |
| rative) | | | | | | | |
| D | Cationic | 0.26 | 16.4 | 15.51 | 20.00 | 4.49 | 36.9 |
| (Compa- | Guar² | | | | | | |
| rative) | | | | | | | |

¹Guar hydroxypropyltrimonium chloride
²Hydroxypropyl guar hydroxypropyltrimonium chloride (double derivative)

As noted from the results given in Table 1, the formulations of this invention using high DS cationic guar gum derivative, i.e., Samples A and B, provided good clarity and wet comb improvement as compared to comparative Sample C which has poor clarity, or compared with comparative Sample D which has poor wet comb. The samples of this invention (A and B) also provided good substantivity without excessive build-up as well as good wet comb.

### EXAMPLE IV

Individual surfactants (amphoteric and non-ionic) were combined in an aqueous solution with the cationic guar gum derivatives of this invention, prepared as described in Example I and identified in Example III as samples A and B. The cationic guar gum derivatives A and B were added in amounts of 0.38% and the surfactants and their amounts are described in Table 2. The samples were evaluated for clarity using a Bausch and Lomb Spectronic 20 spectrophotometer. Results are reported in % transmittance (%T), the higher the % the clearer the formulation.

TABLE 2

| | % Transmittance | | |
|---|---|---|---|
| Sample surfactant | A¹ | B² | Comparative³ |
| (amount active %) | (DS 0.32) | (DS 0.46) | (DS 0.13) |
| Amphoterics | | | |
| Sodium lauroamphodiacetate | 93 | 88 | 68 |
| (8%) | | | |
| Sodium Cocoamphoacetate | 95 | 94 | 91 |
| (6%) | | | |
| Cocamidopropyl betaine (6%) | 96 | 97 | 95 |
| Disodium | 95 | 93 | 90 |
| cocoamphoproprionate (6%) | | | |
| Non-ionics | | | |
| PEG 80 sorbitan laurate (15%) | 62 | 89 | 90 |
| Polysorbate 20 (15%) | 91 | 91 | 81 |

¹Guar hydroxypropyltrimonium chloride
²Guar hydroxypropyltrimonium chloride
³Guar hydroxypropyltrimonium chloride

In the above results, a % transmittance value of 88 or higher is visually clear to the naked eye. The results show that the high DS cationic guar derivatives of this invention (samples A and B) improved clarity with both amphoteric and non-ionic surfactants which are typically used in shampoo compositions. On the other hand, the comparative lower DS cationic derivatives which are commonly used derivatives, are often opaque in these systems.

### EXAMPLE V

A blend of the high cationic guar derivatives of this invention as prepared in Example I with acrylatelamino acrylates/C₁₀₋₃₀ alkyl PEG-20 itaconate copolymer and formulated into a composition described below:

| Ingredients | Parts by weight |
|---|---|
| Deionized water | 70.35 |
| Acrylates/amino acrylates/C₁₀₋₃₀ alkyl | 5.0 |
| PEG-20 itaconate copolymer | |
| Cationic guar (sample A/DS 0.32) | 0.15 |
| Sodium laureth sulfate | 18.0 |
| Cocamidopropyl betaine | 5.5 |
| Sodium hydroxymethyl glycinate | 1 |
| Citric acid to pH of 6-6.5 | qs |

The blended formulation showed excellent feel and foam properties.

What is claimed is:

1. A compatible keratin treating cosmetic composition comprising a cosmetic vehicle, from about 0.1 to 20% by weight of a cationic polygalactomannan derivative which has a cationic group selected from the group consisting of amino, ammonium, imino, sulfonium and phosphonium groups and wherein the cationic content is represented by a degree of substitution (DS) of from about 0.25 to 1.0, and an effective surfactant amount of at least one non-ionic or amphoteric surfactant.

2. The composition of claim 1 wherein the polygalactomannan is guar gum.

3. The composition of claim 2 wherein the cationic group is a tertiary amino or quaternary ammonium ether group.

4. The composition of claim 3 wherein the guar gum has a cationic content represented by a DS of from about 0.25 to 0.6.

5. The composition of claim 3 wherein the cosmetic vehicle is an aqueous system, a solvent system, a mixture of aqueous and solvent systems or an emulsion.

6. A personal cleansing composition comprising the composition of claim 1.

7. The composition of claim 6 wherein the galactomannan is guar gum and the cationic group is a tertiary amino or quaternary ammonium ether group.

8. The composition of claim 7 wherein the cosmetic vehicle is an aqueous system.

9. A shampoo composition comprising the composition of claim 8.

10. The composition of claim 1 wherein the cationic polygalactomannan derivative is prepared by reacting polygalactomannan with a cationic reagent containing a cationic group selected from the group consisting of amino, ammonium, imino, sulfonium and phosphonium groups, the reaction taking place in an alcohol medium and wherein the reaction product is purified by treatment with alkaline base.

11. The composition of claim 10 wherein the polygalactomannan is guar gum.

12. The composition of claim 11 wherein the guar gum is in powder form.

13. The composition of claim 12 wherein the cationic reagent is one containing a tertiary amino or quaternary ammonium ether group.

14. The composition of claim 13 wherein the alkaline base is an alkali metal hydroxide or ammonium hydroxide.

15. The composition of claim 14 wherein the alcohol medium is a monohydric alcohol of 2 to 4 carbon atoms.

16. A personal cleansing composition having the composition of claim 10.

17. A method for preparing high degree of substitution (DS) cationic polygalactomannan derivatives which comprises reacting a polygalactomann with a cationic reagent in an alcohol medium followed by purification of the reaction product by treatment with alkaline base, the cationic reagent containing a cation group selected from the group consisting of amino, ammonium, imino, sulfonium and phosphonium groups and wherein the cationic polygalactomannan has a cationic content represented by a degree of substitution (DS) of from about 0.25 to 1.0.

18. The method of claim 17 wherein the polygalactomannan is guar gum.

19. The method of claim 18 wherein the guar gum is in powder form.

20. The method of claim 19 wherein the alcohol medium is a monohydric alcohol of 2 to 4 carbon atoms.

21. The method of claim 20 wherein the alkaline base is an alkali metal hydroxide or ammonium hydroxide.

22. The method of claim 21 wherein the cationic reagent is one containing a tertiary amino or quaternary ammonium ether group.

23. The method of claim 22 wherein the alcohol is isopropyl alcohol and the alkaline base is sodium hydroxide.
